(19) United States
(12) Patent Application Publication
(10) Pub. No.: US 2013/0085399 A1
(43) Pub. Date: Apr. 4, 2013

(54) THERAPY CONTROL BASED ON NIGHTTIME CARDIOVASCULAR PRESSURE

(71) Applicants: Tommy D. Bennett, Shoreview, MN (US); Yong K. Cho, Maple Grove, MN (US)

(72) Inventors: Tommy D. Bennett, Shoreview, MN (US); Yong K. Cho, Maple Grove, MN (US)

(73) Assignee: Medtronic, Inc., Minneapolis, MN (US)

(21) Appl. No.: 13/250,092

(22) Filed: Sep. 30, 2011

(51) Int. Cl. A61B 5/021; A61N 1/365; A61B 5/0205; A61M 5/168

(52) U.S. Cl. 600/483; 600/485; 604/66; 607/23

(57) ABSTRACT

Techniques for controlling therapy based on a physiological parameter indicative of ventricular filling pressure, such as various cardiovascular pressures, are described. One or more values of the physiological parameter that are collected during nighttime, or while the patient is otherwise asleep, inactive, or within a recumbent position, may be compared to one or more values of the physiological parameter collected during daytime, or while the patient is otherwise awake, active and/or upright. A therapy, such as for treating physiological factors that may lead to worsening HF, may be initiated or adjusted based on the comparison, e.g., if the nighttime values exceed the daytime values.

### TECHNICAL FIELD

The invention relates to patient monitoring and, more particularly, to monitoring cardiovascular pressure.

### BACKGROUND

Patients with chronic congestive heart failure are sensitive to the volume and distribution of fluid throughout the body. Several mechanisms regulate total fluid volume and fluid distribution when the patient is in the upright, supine, and prone positions. As heart failure (HF) progresses, blood volume may increase in some regions and the patient may experience venous constriction and decreased venous compliance, resulting in increased blood pressure and symptoms of HF. Variations in patient position, e.g., standing, sitting, and lying prone or supine, may trigger one or more mechanisms affecting the distribution of fluid within the body, further exacerbating the symptoms of HF, such as pulmonary edema, nocturia, and dyspnea. Additionally, changes in the autonomic nervous system, such as sympathetic activity or circulating catecholamines, may result is redistributions of fluid volume within the body, also contributing to symptoms of HF.

Treatment for HF may focus on the underlying factors leading to the symptoms of HF before the symptoms of HF manifest or worsen, while simultaneously correcting any life style factors, such as smoking or hypertension, contributing to the condition. To treat factors contributing to HF, a clinician may prescribe a mix of medications and therapies. For example, medication may reduce cardiac filling pressure by reducing fluid volume within the body of the patient (e.g., diuretics) or by reducing the constriction of the vasculature (e.g., vasodilators and protein inhibitors).

Example diuretics include, but are not limited to, loop diuretics such as furosemide and torsemide, metolazone, thiazide, and other potassium sparing diuretics, such as spironolactone. Therapeutic techniques for treating factors that could cause new onset or worsening of HF may include other techniques for removing excess fluid volume, such as by ultrafiltration (aquaphoresis). The effects of vasoconstriction may be countered with a vasodilator or the administration of an inhibitor agent, such as angiotensin-converting inhibitors, preventing the activation of various enzymes the body produces to increase blood pressure in response to HF. Some medications, such as nitroglycerin (vasodilator), are used in response to acute symptoms while other medications and therapies may be administered over an extended period as part of an ongoing course of treatment.

### SUMMARY

Most typically, HF patients and other human subjects have increases in ventricular filling pressure during the active daytime hours, due to mechanisms acting to adjust filling pressures to accommodate the cardiovascular stresses encountered with normal activities of daily life. These increases in daytime filling pressures are typically seen, even though gravitational forces associated with upright body position, taken alone, will be acting to decrease filling pressures as fluid shifts away from the thoracic vasculature to the gravity dependent body areas like the gut and lower extremities. However, some HF patients show atypical patterns of circadian filling pressures, and more particularly experience nighttime ventricular filling pressures that are higher than their active daytime filling pressures. Elevated nighttime filling pressure may produce a variety of undesired patho-physiological responses, such as increased load on the heart (left and right ventricles), increased filtration of fluid to extravascular compartments (pulmonary congestion or edema), and chronic changes in pulmonary vascular reactivity.

Nighttime symptoms of HF, while common, often do not become sufficiently severe for the patient to notice, e.g., to wake the patient or hinder patient sleep, until HF has significantly worsened. Additionally, the patient must communicate these symptoms to a clinician to enable the clinician to diagnose whether the patient is experiencing changing or excessive symptoms.

In general, this disclosure describes techniques for monitoring and treating the physiological changes that may lead to manifestation or worsening symptoms of HF. By monitoring the patient, a history of physiological values may be promptly available for clinical examination. Such monitoring of the patient may also allow prompt, e.g., automatic, adjustment of the therapy for the treatment of the changes that might otherwise lead to new or worsening HF symptoms.

More particularly, the disclosure describes techniques for monitoring physiological parameters, such as cardiovascular pressures indicative of ventricular filling pressure. One or more values of the physiological parameter that are collected during nighttime, or while the patient is otherwise asleep, inactive, or within a recumbent position, may be compared to one or more values of the physiological parameter collected during daytime, or while the patient is otherwise awake, active and/or upright. A therapy for treating factors contributing to HF may be initiated or adjusted based on the comparison, e.g., if the nighttime values exceed the daytime values. For example, a medication dispenser or drug pump may be automatically directed to provide more medication, e.g., during nighttime, if the nighttime values exceed the daytime values. Since the patients are supine and inactive at night, delivery of additional vasodilator therapy during this time may particularly be considered, since it is the period of time when filling pressures are most dramatically elevated and the risk of symptomatic systemic hypotension is lower at this time.

In one example, a system comprises a sensor configured to measure a plurality of values of a physiological parameter indicative of ventricular filling pressure of a patient, and a processor. The processor is configured to, for each of the measured values of the physiological parameter, categorize the value as one of a daytime value or a nighttime value, compare one or more of the daytime values to one or more of the nighttime values and, if the nighttime values are larger than the daytime values, direct a modification of delivery of a therapy for treatment of heart failure.

In another example, a method comprises measuring a plurality of values of a physiological parameter indicative of ventricular filling pressure of a patient by a sensor. The method further comprises, with a processor, for each of the measured values of the physiological parameter, categorizing the value as one of a daytime value or a nighttime value, comparing one or more of the daytime values to one or more of the nighttime values; and, if the nighttime values are larger than the daytime values, directing a modification of delivery of a therapy for treatment of heart failure.

In another example, a system comprises means for measuring a plurality of values of a physiological parameter indicative of ventricular filling pressure of a patient, means for, for each of the measured values of the physiological parameter, categorizing the value as one of a daytime value or a nighttime value, means for comparing one or more of the daytime values to one or more of the nighttime values, and means for, if the nighttime values are larger than the daytime values, directing a modification of delivery of a therapy for treatment of heart failure.

In another example, a system comprises a sensor configured to measure a plurality of values of a physiological parameter indicative of ventricular filling pressure of a patient, and a processor. The processor is configured to for each of the measured values of the physiological parameter, categorize the value as one of a daytime value or a nighttime value, compare one or more of the daytime values to one or more of the nighttime values, and direct a modification of delivery of a therapy to the patient based on the comparison.

The details of one or more examples are set forth in the accompanying drawings and the description below. Other features, objects, and advantages will be apparent from the description and drawings, and from the claims.

### BRIEF DESCRIPTION OF DRAWINGS

FIGS. 1A and 1B are conceptual diagrams illustrating example systems that monitor a physiological parameter of the patient and control treatment of HF.

FIG. 2 is a block diagram illustrating an example system to monitor a physiological parameter of the patient and control treatment of HF.

FIG. 3 is a flow diagram illustrating an example operation of a pressure monitor for monitoring a physiological parameter of the patient and controlling treatment of HF.

FIG. 4 is a flow diagram illustrating an example method of monitoring a physiological parameter of a patient and controlling treatment of HF.

FIG. 5 is a flow diagram illustrating an example method of controlling treatment for HF based on comparing measured values of a physiological parameter.

FIG. 6 is a chart illustrating trends of physiological parameters for an example patient exhibiting an atypical pattern of circadian filling pressures.

### DETAILED DESCRIPTION

An increase in ventricular filling pressure is characteristic of HF. During HF, one or more of the ventricles may stiffen and lose some of the ability to fill or to empty (contract) normally. In order to maintain stroke volume, i.e., the volume of blood pumped with each beat of the heart, the affected ventricle expands and begins to operate at a higher pressure. Further reductions in ventricular performance may lead to further reductions in cardiac output, fluid redistribution within the body, or additional fluid retention by the body, thus further increasing filling pressures and worsening the HF.

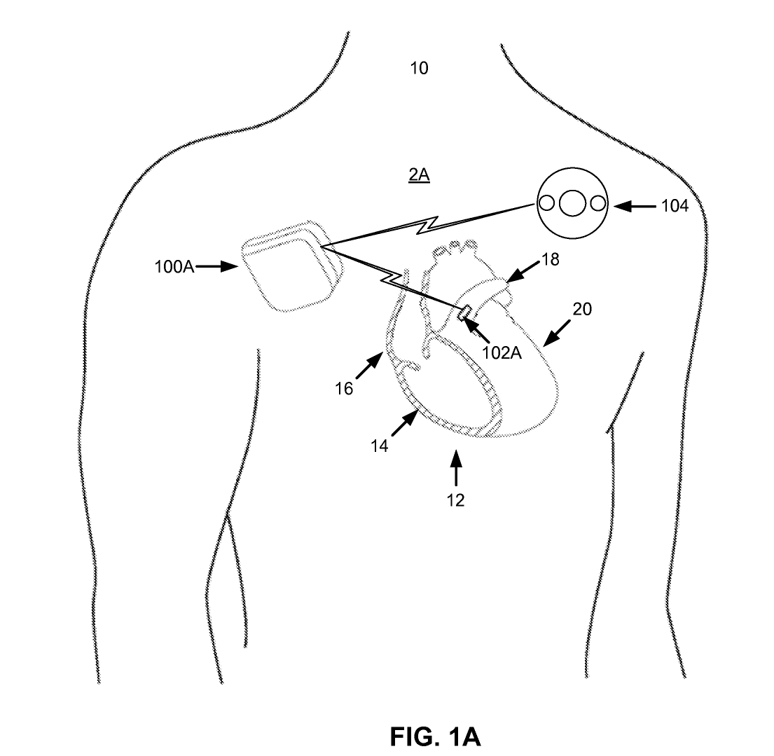

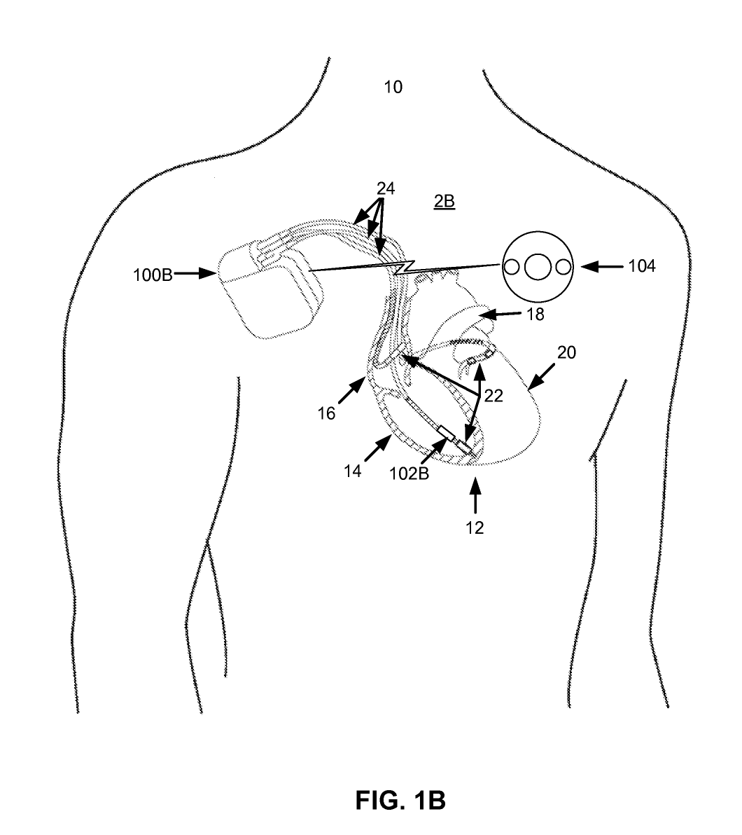

FIGS. 1A and 1B are conceptual diagrams illustrating example systems 2A and 2B that monitor a physiological parameter indicative of the ventricular filling pressure of patient 10, and control the treatment of HF. FIG. 1A depicts example system 2A comprising pressure monitor 100A, sensor 102A, and automated medication dispenser (AMD) 104. The system monitors a physiological parameter indicative of the ventricular filling parameter of heart 12 of patient 10.

In some examples, as illustrated in FIGS. 1A and 1B, a pressure monitor may comprise an implantable medical device (IMD). In other examples, a pressure monitor may be located externally, e.g., on the body of patient 10 or proximate to patient. A pressure monitor may be an independent device located separately from the other devices in the system for monitoring a physiological parameter indicative of the ventricular filling pressure in the body of patient 10. Alternatively, one or more of an AMD or a sensor may be connected to or incorporated into the pressure monitor. A pressure monitor may comprise an antenna to establish wireless communications with one or more devices, or a port to form a wired connection to one or more devices.

Sensors 102 may be located in the pulmonary artery of patient 10. Other locations for sensors 102 may include right ventricle 14, left ventricle, and right atrium, of heart 12 as well as the vasculature of patient 10, such as the central veins, the peripheral veins, arteries, or other locations where measures indicative of fluid volume status of the patient may be obtained. For measurement of arterial pressure, for example, sensors 102 may be placed in or next to an artery, such as the femoral, brachial, subclavian, or other artery.

In examples in which the pressure monitor comprises an IMD, the pressure monitor may comprise, for example, an implantable monitor, or an IMD that additionally provides therapy, such as a cardiac electrical stimulus device, implantable drug delivery device (pump), or the like. A cardiac electrical stimulus device may be an implantable pacemaker, implantable cardioverter-defibrillator, implantable pacemaker-cardioverter-defibrillator, cardiac resynchronization therapy (CRT) device, or implantable neurological stimulator. In the illustrated example, pressure monitor 100A comprises an implantable monitor. For example, pressure monitor 100A may comprise an implantable monitor that includes electrodes and/or one or more other sensors configured to monitor one or more physiological parameters of patient 12 in addition to pressure, such as physiological parameters derivable from a cardiac electrogram detected via electrodes formed on or integral with a housing of pressure monitor 100A. In some examples, pressure monitor 100A may comprise a Reveal® monitor, commercially available from Medtronic, Inc. of Minneapolis, Minn., which has been configured to perform the techniques described herein.

Pressure monitor 100A may communicate with sensor 102B and AMD 104. This communication is depicted as wireless, but in other examples may comprise one or more wired connections between pressure monitor 100A and sensor 102A or AMD 104. In some examples, pressure monitor 100A may establish communication links with one or more external devices, e.g., clinician or first responder monitoring equipment. Communication with external devices may occur via radio-frequency or proximal inductive media, or over a cellular or wireless internet network. Information transmitted to external devices may comprise notifications that abnormal patterns of physiological parameters have been measured, emergency alerts, and recorded history of measured physiological parameters. Similarly, pressure monitor 100A may receive updated instructions from a clinician programmer or similar device.

Pressure monitor 100A may receive data indicating the value of a physiological parameter that is indicative of ventricular filling pressure from sensor 102A. Pressure monitor 100A may control the sampling of the physiological parameter by sensor 102A. For example, sensor 102A may measure the value of the physiological parameter when instructed to or scheduled by pressure monitor 100A. In other configurations, sensor 102A may sample the physiological parameter at a fixed interval or on a preset schedule and transmit the measured value of the physiological parameter to pressure monitor 100A.

Pressure monitor 100A may determine whether the measurements made by sensor 102A occurred during a daytime or nighttime period, and accordingly sort the measurements into daytime measurements and nighttime measurements. Pressure monitor 100A may include a real-time clock, and may determine whether a measurement was made during a daytime period or nighttime period based on the real-time clock. In some examples, a clinician or other user may provide instructions regarding the daytime and nighttime periods to pressure monitor 100A, e.g., via an external programming device (not shown) in communication with pressure monitor 100A.

In some examples, pressure monitor 100A may determine whether a measurement is a daytime or nighttime measurement based on whether patient 10 is upright, active and/or awake, or recumbent, inactive and/or asleep. Pressure monitor 100A may include or be coupled to one or more sensors that generate a signal indicative of the posture and/or activity of the patient to determine whether a measurement is a daytime or nighttime measurement. For example, pressure monitor 100A may include or be coupled to one or more accelerometers that generate signals indicative of posture and/or activity of patient 10. As another example, pressure monitor 100A may include or be coupled to electrodes which may detect a cardiac electrogram, from which heart rate may be determined, or a signal indicative of respiration from which a respiration rate may be determined, both of which may indicate the activity level of patient 10. Although the terms “daytime” and “nighttime” are used to describe two periods in which cardiovascular pressures are measured and from which cardiovascular pressure measurements are compared, it is understood that the daytime and nighttime measurements do not necessarily occur during daylight or at night, respectively.

Pressure monitor 100A compares the measured values of a physiological parameter indicative of the ventricular filling pressure, e.g., measured cardiovascular pressures, to determine if the ventricular filling pressure is increasing during a time period when patient 10 is likely in a recumbent position, e.g., at night. Pressure monitor 100A may calculate a representative statistic, such as the mean or median, of the measured values of the physiological parameter for all or a portion of the daytime and nighttime periods. Pressure monitor 100A may compare the representative statistics for the daytime and nighttime periods to determine if the ventricular filling pressure is increasing during the night.

If pressure monitor 100A determines that the ventricular filling pressure of heart 12 of patient 10 is greater at night, pressure monitor 100A may take one or more actions. Possible actions include initiating or adjusting a therapy, such as ultrafiltration, pacing, CRT, or administration of a medication, or communicating with the patient or clinician, alerting them to the potential problem. In some situations, pressure monitor 100A may be programmed to take no action when the night time ventricular filling pressure is higher than the day time filling pressure, such as when ventricular filling pressure, though higher during the nighttime period than the daytime period, is below a threshold.

Sensor 102A may comprise a pressure sensor, e.g., a capacitive pressure sensor, implanted in the vasculature or heart 12 of patient 10. For example, sensor 102 may be located within the pulmonary artery 18 of patient 10, as shown in FIG. 1A, or affixed to the wall of right ventricle 14 of heart 12. Alternative example systems may include an electrical impedance sensor or a heart sound monitor, which may generate signals that vary as a function of ventricular or other cardiovascular pressures, as sensor 102A instead of or in addition to a pressure sensor.

AMD 104 may automatically dispense a medication to treat factors contributing to HF in patient 10. AMD 104 may be implanted into the body of patient 10, e.g., may be an implanted pump, or may be external to patient. Examples of external medication dispensers include external pumps or external pill dispensers. The dosage of medication or therapy administered by AMD 104 may be adjusted by pressure monitor 100 based on the comparison of the daytime and nighttime ventricular filling pressures, or measured values of physiological parameters indicative thereof.

In some examples, in addition to or as an alternative to controlling AMD 104, pressure monitor 100A may take other actions in response to determining that nighttime ventricular filling pressures exceed daytime ventricular filling pressures. For example, pressure monitor 100A may communicate with an external computing device, such as medical device programmer, personal computer, or cellular telephone, to notify the patient, a physician, or another caregiver of the worsening HF of the patient. As another example, pressure monitor 100A may communicate with such devices to instruct the patient, physician or caregiver to modify a dosage of a medication administered to the patient. As another example, pressure monitor 100A may communicate with another implanted or external medical device that administers another treatment, such as ultrafiltration, pacing, or CRT, to initiate or modify the treatment to treat the factors contributing to HF in patient 10.

FIG. 1B depicts another example system 2B comprising a pressure monitor 100B, sensor 102B, and AMD 104. As was the case with system 2A, system 2B monitors a physiological parameter indicative of the ventricular filling pressure of heart 12 of patient 10. In general, the components of system 2B may be similar to, and provide similar functionality to, the like components of system 2A.

In the illustrated example, pressure monitor 100B may comprise an implantable pacemaker, cardioverter-defibrillator, or pacemaker-cardioverter-defibrillator, which may also provide CRT. Leads 24 may connect pressure monitor 100B to one or more electrodes 22 located in various portions of heart 12 of patient 10. For example, one or more electrodes 22 may be located in right atrium 16, right ventricle 14, and/or proximate to left ventricle 20.

In the illustrated example, sensor 102B is connected to pressure monitor 100B by one of leads 24. In the illustrated example, sensor 102B is located in right ventricle 14 of heart 12. Sensor 102B may comprise a pressure sensor, e.g., a capacitive pressure sensor, which may detect a cardiovascular pressure indicative of ventricular filling pressure, such as systolic, diastolic, or mean right-ventricular pressure, or estimated pulmonary artery diastolic pressure.

Pressure monitor 100B may utilize electrodes 22 to monitor one or more cardiac electrograms of heart 12, determine cardiac or thoracic impedance levels, e.g., for monitoring of fluid accumulation, and/or to deliver pacing and/or defibrillation therapy in the event of a detected cardiac arrhythmia. As described above with respect to pressure monitor 100A, pressure monitor 100B may determine whether measurement made by sensor 102B is a daytime or nighttime measurement based on patient posture and/or activity. In some examples, pressure monitor 100A may determine patient activity level based on a heart rate derived from a cardiac electrogram signal sensed via electrodes 22, or based on other signals sensed via electrodes 22 or generated by other sensors, as described above. Pressure monitor 100B may maintain one or more wireless connections to various devices, e.g., AMD 104 or clinician monitoring equipment.

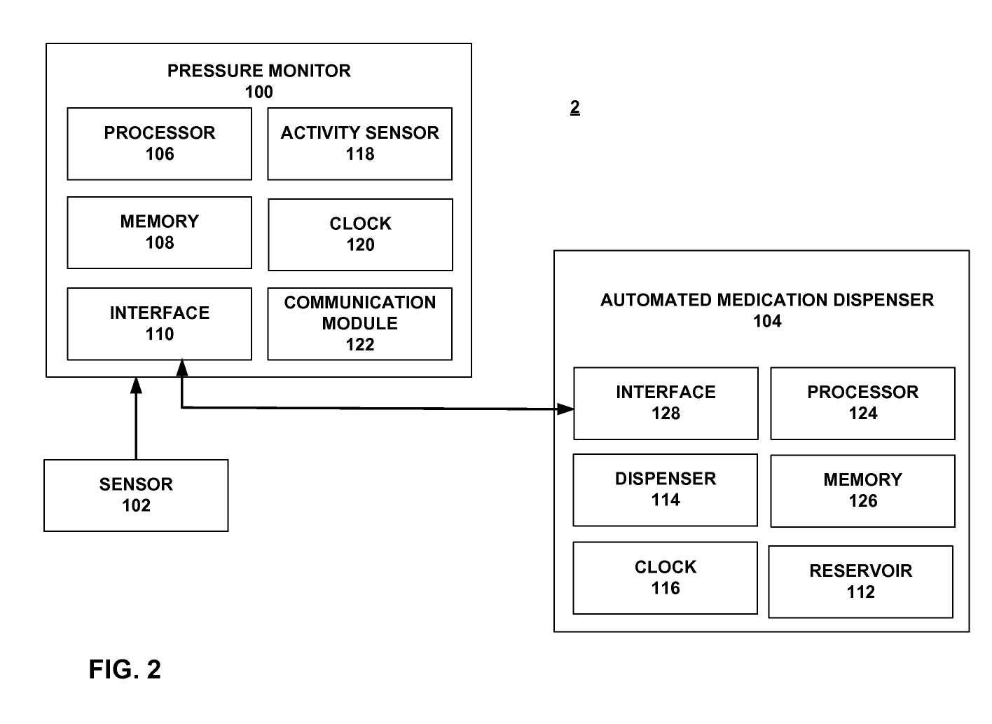

FIG. 2 is a block diagram illustrating an example system 2 to monitor a physiological parameter of patient 10 and control treatment of HF. In the illustrated example, system 2 comprises a pressure monitor 100, sensor 102, and AMD 104, which may correspond generally to the pressure monitors 100A and 100B, sensors 102A and 102B, and medication dispenser 104 of FIGS. 1A and 1B. Pressure monitor 100 may comprise processor 106, memory 108, interface 110, activity sensor 118, clock 120 and communication module 122. AMD 104 may comprise reservoir 112, dispenser 114, clock 116, processor 124, memory 126, and interface 128.

Example system 2 depicted in FIG. 2 is configured to monitor the ventricular filling pressure of patient 10, and may adjust or initiate treatment for the factors contributing to HF automatically in the event that an abnormal pattern of ventricular filling pressures are detected. Sensor 102 may monitor the ventricular filling pressure or a physiological parameter indicative of the ventricular filling pressure, and communicate the measurements to pressure monitor 100. Pressure monitor 100 may analyze the data gathered by sensor 102, and determine a course of action based on that analysis. For example pressure monitor 100 may communicate a change in dosage to AMD 104, or direct AMD 104 to deliver an otherwise unscheduled dose of medication.

Example system 2 may administer any of a variety of therapies and/or medications, such as described herein. For example, AMD 104 may dispense a venodilator, or other vasodilator (such as nitroglycerin), or an enzyme inhibitor, such as angiotensin-converting inhibitors. These medications may be dispensed according to a fixed schedule or in response to the detection of elevated filling pressures by sensor 102. In some examples, AMD 104 or another implanted or external device may administer a therapy other then drug delivery, such as CRT or ultrafiltration. In some examples, pressure monitor 100 may communicate with patient 10 or an external device, such as an automatic pill dispenser, to regulate the consumption of medication, such as oral pills, by patient 10.

Pressure monitor 100 may be configured to receive the measurements made by sensor 102, store the measurements, compare the measured values or a representation thereof, and initiate at least one action based on the comparison. Pressure monitor 100 may be implemented as an independent device, or incorporate one or more treatment devices, such as an implantable pacemaker and/or defibrillator, which in some cases may provide CRT.

Processor 106 of pressure monitor 100 may control the operation of pressure monitor 100 and, through communication with sensor 102 and AMD 104, the operation of system 8. Processor 106 may control interface 110 to receive or transmit data and commands from sensor 102 or automated medical dispenser 104. Processor 106 may perform read and write operations to memory 108, storing measured values of a physiological parameter indicative ventricular filling pressure obtained from sensor 102 in memory 108 and retrieving the data to perform comparisons. Processor 106 may compare the daytime and nighttime ventricular filling pressures to determine if an abnormal circadian trend in the ventricular filling pressure exists. Based on the comparison of the ventricular filling pressures, processor 106 may determine an appropriate action to initiate, which may include communicating with the clinician or patient 10, or adjusting the dosage of medication provided to patient 10 by automated medication dispenser 104, as examples.

Processor 106 may compare daytime and nighttime ventricular filling pressures, or measured values of physiological parameters representative thereof. Processor 106 may compute a representative metric of the values over a time period. For example, processor 106 may calculate the mean or median of the measured values occurring over the preceding “day” and “night”.

Day and night may be defined by a pre-set time period or may be determined using an activity level and/or posture sensor. In some examples, each measurement received by pressure monitor 100 may be associated with a measured activity level, such that measurement may be determined to be made at ‘night’ when the measurement was made during an extended period of inactivity and vice-versa. Processor 106 may, in some configurations, perform comparisons of multiple time periods occurring within one or more day/night cycles. This may allow processor 106 to evaluate long-term trends in progression of the HF of patient 10, and may allow a clinician to better observe the effect of the prescribed course of treatment.

Based on the comparison of the day and night ventricular filling pressures, processor 106 may initiate an action. The action may comprise alerting patient 10 or the clinician to the presence of an abnormal trend or other dangerous condition via text message, email, internet message, audible signal, vibration, or similar mechanism. In some examples, an alert may be delivered by communication between pressure monitor 100 and one or more external devices via communication module 122.

In some examples processor 106 may adjust the treatment being administered for the heart failure of patient 10. For example, processor 106 may instruct AMD 104 to incrementally increase the dosage of a medication, or AMD 104 or another device to incrementally increase the intensity of another therapeutic treatment, being administered to patient 10 based on the nighttime pressure being greater than the daytime pressure. In some examples, processor 106 may adjust the dosage based on a predefined relationship between the measured values of the physiological parameter and the dosage the medication being dispensed, e.g., between the nighttime values and dosage, or between the difference between or ratio of nighttime and daytime values of the physiological parameter indicative of ventricular filling pressure Such a relationship may be stored in memory 108, and thus be available to processor 106, of pressure monitor 100. Processor 106 may also be configured to temporarily or permanently reduce or eliminate the treatment being applied by AMD 104. For example, processor 106 may be configured to periodically temporarily decrease the dosage or intensity of the treatment of patient 10 to verify that a previously-increased level of treatment due to a comparison of nighttime and daytime ventricular filling pressures is required by the condition of patient 10.

Processor 106 may determine whether a measurement made by sensor 102 should be considered a daytime measurement or a nighttime measurement by referencing clock 120 or activity sensor 118, for example. For example, daytime and nighttime may be defined by a set time period programmed by patient 10 or a clinician, e.g., by setting the period from 11:00 p.m. to 6:00 a.m. as indicated by clock 120 as night, and the remainder of each day as daytime.

In some examples, processor 106 may consult data provided by activity sensor 118 that was contemporaneous with measurements by sensor 102 to determine whether the measurements are daytime measurements or nighttime measurements. The data provided by activity sensor 118 may indicate the activity level and/or posture of patient 10 when, or proximate to when, the measurement indicative of ventricular filling pressure was made by sensor 102. Processor 106 may determine that a measured value from sensor 102 is a nighttime measurement if the data from activity sensor 118 indicates that the patient was lying down, or that the measurement occurred during an extended period of low activity level, e.g., as would occur when patient 10 sleeps. Conversely, processor 106 may determine that a measured value from sensor 102 is a daytime measurement if the data from activity sensor 118 indicates that the patient was upright, or that the measurement occurred during a period of relatively higher activity level, e.g., associated with activity of daily living. In some examples, processor 106 may determine whether a physiological parameter measurement made by sensor 102 is a daytime or nighttime measurement based on activity sensor 118, clock 120, or both activity sensor 118 and clock 120, e.g., measurements are classified as nighttime if they occur during a particular period of the day and during a period in which the activity and/or posture of the patient was consistent with sleeping.

Memory 108 may store the values of a physiological parameter indicative of ventricular filling pressure received from sensor 102 via interface, and associated data, including whether processor 106 identified the measurement as a daytime or nighttime measurements. Memory 108 may also store other data, such as the time the measurement was made or the output of activity sensor 118 contemporaneous with the measured value from sensor 102.

Activity sensor 118 may detect the activity level and/or position of patient 10. In some examples, activity sensor 118 may comprise one or more accelerometers, e.g., such as a 3-axis accelerometer. In some examples, activity sensor 118 may comprise a heart rate monitor, which may comprise, for example, electrodes for detected an EGM signal. During the daytime time period, patient 10 is likely active and would exhibit an increased heart rate when compared with the nighttime time periods when patient 10 is at rest. Activity sensor 118 may be incorporated into the housing of pressure monitor 100 or may be located separately from pressure monitor 100.

Clock 120 may allow processor 106 to determine the time at which a measurement made by sensor 102 was made as well as synchronizing the operations of pressure monitor 100. In some configurations, day and night time periods may be predefined or customized for a specific patient. Processor 106 may use time information supplied by clock 120 to sort measurements received from sensor 102 into day or night categories and store that information along with the measured values and the time the measurements were made in memory 108. The time supplied by clock 120 may also be used by processor 106 to schedule measurements by sensor 102 or adjust dosage levels by AMD 104.

Processor 106 comprises any suitable arrangement of hardware, alone or in combination with software and/or firmware, to perform the techniques attributed to processor 106 and pressure monitor 100. In various examples, processor 106 can include any one or more microprocessors, digital signal processors (DSPs), application specific integrated circuits (ASICs), field programmable gate arrays (FPGAs), or any other equivalent integrated or discrete logic circuitry, as well as any combinations of such components.

Processor 106 may store measured values of a physiological representative of ventricular filling pressure obtained by sensor 102 in memory 108. Memory 108 may also store the time, patient posture, or patient activity level at which the measurement was made. Memory 108 may retain a history of measurements made by sensor 102, allowing a clinician or other practitioner to better evaluate the effectiveness of treatment and the progression of the HF of patient 10 over time.

Memory 108 may include any volatile or non-volatile media, such as a random access memory (RAM), read only memory (ROM), non-volatile RAM (NVRAM), electrically erasable programmable ROM (EEPROM), flash memory, and the like. Memory 108 may store instructions for execution by processor 106 that cause processor 106 to perform the techniques attributed to processor 106 and pressure monitor 100 herein.

Interface 110 allows pressure monitor 100 to communicate with and control sensor 102 and/or AMD 104. Interface 110 may transmit command signals from processor 106 to linked devices and data from linked devices to processor 106. Interface 110 may comprise one or more of a radio wireless transceiver and antenna, a wired access port, a fiber-optic access port, or a transceiver for other types of wireless communication.

Communications module 122 may provide a radio-frequency or other network communications interface with devices external to patient 10, e.g., a clinician monitor or server located apart from patient 10. In some examples, communications module 122 may alert patient 10 to changes or abnormal patterns in the ventricular filling pressure. Such notifications may include vibration, audible alerts, text messages sent to a telecommunications device of patient 10, or a prerecorded message. Following the detection of an abnormal pattern or emergency communications module 122 may also automatically transmit some or all of the stored measured values of a physiological parameter indicative of ventricular filling pressure to a clinician, first responder, or admitting hospital, allowing appropriate diagnosis of the condition of patient 10 to be expedited.

Sensor 102 may measure one or more physiological parameters representative of the ventricular filling pressure of heart 12 of patient 10. Sensor 102 may be configured to measure the one or more physiological parameters on command from pressure monitor 100 or periodically based on a fixed time interval or preset schedule. Sensor 102 may transmit the one or more measured values to pressure monitor 100 for processing, e.g., by processor 106, and storage, e.g., within memory 108. In some examples, multiple sensors 102 may be implanted in patient 10, allowing pressure monitor 100 to monitor multiple physiological parameters indicative of the ventricular filling pressure of heart 12 of patient 10. In other examples, a single sensor 102 may be used.

In some configurations, sensor 102 may comprise a pressure sensor implanted in the vasculature of patient 10, or in heart 12, e.g., a ventricle of heart 12, of patient 10. Implanted in a ventricle, sensor 102 may directly measure the ventricular filling pressure, e.g., a mean ventricular pressure or diastolic ventricular pressure. In other locations in the vasculature, the measured value of the blood pressure may indicate the ventricular filling pressure. For example, a mean or diastolic pulmonary artery pressure may indicate ventricular filling pressure.

Alternatively, sensor 102 may comprise a microphone implanted within the torso of patient 10 and be configured to monitor heart sounds. Monitoring the sounds of contraction, valve activity, and blood flow within heart 12 of patient 10 may indicate of the pressure of blood within heart 12. In other examples, sensor 102 may comprise one or more electrodes arranged around the heart or other locations about the body of patient 10. These electrodes may be configured to measure the intrathoracic, cardiac, or vasculature impedance. These measures of impedance may be inversely related to the amount of fluid in various regions of the body and, thus, the ventricular filling pressure.

AMD 104 may dispense medication to treat HF in patient 10. AMD 104 may dispense or titrate medication according to a predefined dosage schedule, e.g., based on instructions in memory 126 and executed by processor 124, or under the control of a second device, such as pressure monitor 100, via interface 128. Pressure monitor 100 may be able to adjust the dosage amounts or frequency of the medication administered by AMD 104 based on a comparison of the daytime and nighttime ventricular filling pressures. AMD 104 may be implanted in patient 10 or carried externally.

Reservoir 112 may contain medication to be dispensed by AMD 104. AMD 104 may comprise one or more reservoirs 112, each of which may contain a different medication. Reservoir 112 may be accessible to a clinician or other authorized person to allow the replenishment of the medication contained in reservoir 112, e.g., reservoir 112 may have port allowing a hollow needle to penetrate into reservoir 112 opening fluid communication between reservoir 112 and an external store of medication. The level of medication within reservoir 112 may be monitored, e.g., by processor 124, and the medication level may be transmitted through interface 128 to pressure monitor 100 for communication to patient 10 or a clinician, e.g., via communication module 122 of patient monitor 100.

Dispenser 114 may be configured to administer a dosage of medication under the control of processor 124, e.g., according to a fixed schedule stored in memory 126, or at the command of a second device, such as pressure monitor 100, via interface 128. Dispenser 114 may draw the medication to be dispensed from reservoir 112. The schedule or dosage of medication and/or therapy may be adjusted remotely, e.g., via a signal from pressure monitor 100, or according to instructions in memory 126. Dispenser 114 may supply any therapy that reduces the ventricular filling pressure of heart 12 of patient 10, such as vasodilators and diuretics.

AMD 104 may comprise clock 116. Clock 116 may be used to synchronize the operations of processor 124 and dispenser 114, and enable processor 124 to control dispenser 114 to administer therapy to treat the HF of patient 10 at specific times or at set intervals.

Processor 124 comprises any suitable arrangement of hardware, alone or in combination with software and/or firmware, to perform the techniques attributed to processor 124 and AMD 104. In various examples, processor 124 can include any one or more of microprocessors, digital signal processors (DSPs), application specific integrated circuits (ASICs), field programmable gate arrays (FPGAs), or any other equivalent integrated or discrete logic circuitry, as well as any combinations of such components.

Processor 124 may control the operation of AMD 104. For example, instructions specifying the administration of a medication by AMD 104 may be stored in memory 126 and executed by processor 124. Processor 124 may cause dispenser 114 to dispense medication based on the time kept by clock 116. In other examples, processor 124 may administer, or adjust the administration of therapy, based on instructions received from a second device, such as pressure monitor 100, received via interface 128. Processor 124 may store data, such as a history of the administration of therapy to patient 10 in memory 126. Processor 124 may also alter or replace instructions for the dispensing of medication by AMD 104 in memory 126 in response to, for example, instructions from a clinician or pressure monitor 100. Processor 124 may transmit some or all of the contents of memory 126 to a second device, such as pressure monitor 100, through interface 128.

Memory 126 may include any volatile or non-volatile media, such as a random access memory (RAM), read only memory (ROM), non-volatile RAM (NVRAM), electrically erasable programmable ROM (EEPROM), flash memory, and the like. Memory 126 may store instructions for execution by processor 124 that cause processor 124 to perform the techniques attributed to processor 124 herein.

Interface 128 allows AMD 104 to communicate with pressure monitor 100. Interface 128 may receive command signals from processor 106 of pressure monitor 100 and transmit data, such as medication levels in reservoir 112 or power available to AMD 104, regarding the status of AMD 104 to pressure monitor 100. Processor 124 may control interface 128. Interface 128 may comprise one or more of a radio wireless transceiver and antenna, a wired access port, a fiber-optic access port, or a transceiver for other types of wireless communication.

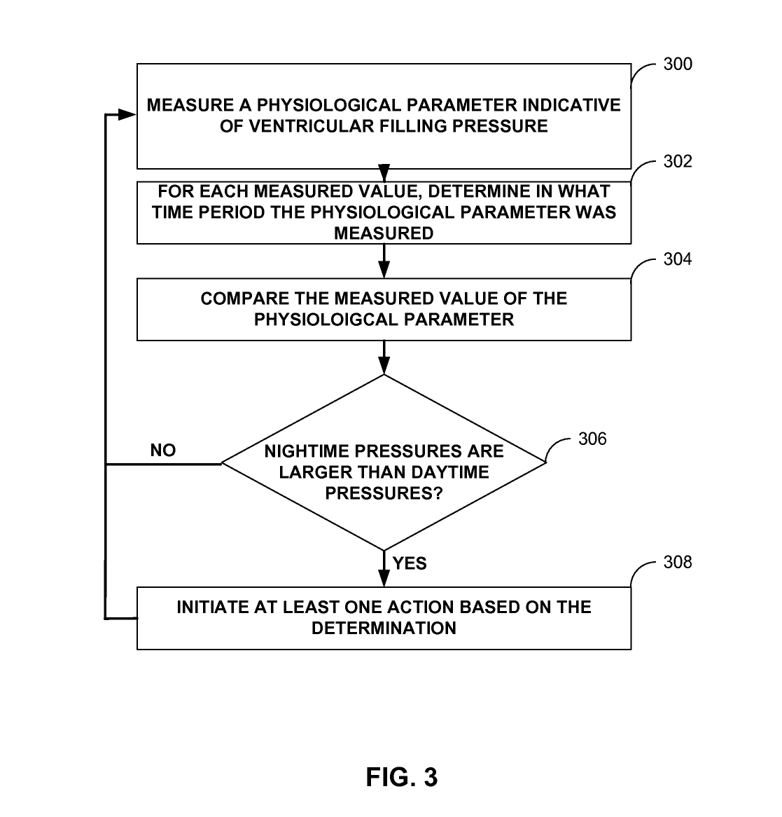

FIG. 3 is a flow diagram illustrating an example operation of pressure monitor 100 for monitoring a physiological parameter of patient 10 and controlling treatment of HF. The example method includes measuring a physiological parameter indicative of ventricular filling pressure (300); for each of the measured values, determining in what time period the physiological parameter was measured (302); comparing the measured value of the physiological parameter (304); determining if the ventricular filling pressure was larger at night or during the day (306); and initiating at least one action based on the determination (308).

Sensor 102 measures a physiological parameter indicative of ventricular filling pressure (300) and transmits the measured value to pressure monitor 100. The physiological parameter may be the ventricular filling pressure itself, as measured by a pressure sensor implanted in one or both ventricles of heart 12 of patient 10. In some examples, sensor 102 may additionally or alternatively measure heart sounds generated during the cardiac cycle, blood pressures measured elsewhere in the vasculature of patient 10, or electrical impedances across heart 12 or the torso of patient 10. Sensor 102 may perform these measurements on a schedule or based on commands from pressure monitor 100. Multiple sensors 102 may be implanted into patient 10 and may measure one or more of the aforementioned physiological parameters.

Upon or after receipt of the measured values from sensor 102, processor 106 of pressure monitor 100 may store the measured values of the physiological parameter in memory 108. Processor 106 may also store time and day/night and/or activity level information along with the measured value. Memory 108 may retain an amount of data, e.g., 24 or 48 hours of data, allowing pressure monitor 100 to compare daytime and nighttime values of the physiological parameter, and thereby compare daytime and nighttime ventricular filling pressures. In other embodiments, memory 108 may store an extensive history of measured values of the physiological parameter, allowing a clinician to observe long terms in the progression of the HF of patient 10, potentially allowing the clinician to provide a better evaluation of the effectiveness of treatment.

For each of the measured values, processor 106 may determine in what time period the physiological parameter was measured by sensor 102, e.g., whether the measurement is a daytime measurement or a nighttime measurement (302). In some examples, pressure monitor 100 may use time data supplied by clock 120 to determine when the physiological parameter was measured and sort measured values based on predefined daytime and nighttime periods. In some examples, processor 106 may compare activity level or position data supplied by activity sensor 118 to thresholds, criteria or other predetermined characteristics of daytime and nighttime periods. An increased activity level or typically upright position indicates that patient 10 may be awake, and may thus be associated with a daytime measurement. Periods of reduced activity or typically recumbent positions indicate that patient 10 is at rest, and may thus associated with nighttime measurements. In other examples, processor 106 may determine night and day periods by consulting clock 120 in conjunction with activity levels or posture measured by activity sensor 118. In some examples, pressure monitor 100 may monitor activity levels for one or more day/night cycles to determine the general routine of patient 10. The general routine of patient 10 may be used to define day and night time periods when the patient is likely upright or prone/supine. Future measurements of a physiological parameter indicative of ventricular filling pressure may be assigned to a day or night time period based on the generic day and night time periods without further consulting the activity levels of patient 10.

Processor 106 may compare the measured values of the physiological parameter made during the daytime and nighttime time periods (304). Processor 106 may retrieve measured values of the physiological parameter from memory 108 and calculate a representative metric, such as a mean or median, for the data set of measured values in a given time period. Processor 106 may then compare the representative values of the day and night time periods to determine if the ventricular filling pressure was larger at night or during the day (406). If the nighttime ventricular filling pressures are less than the daytime filling pressures, therapy may, in some examples, not be altered, and pressure monitor 100 and sensor 102 may continue to measure a physiological parameter indicative of the ventricular filling pressure (300). On the other hand, processor 106 may cause pressure monitor 100 or AMD 104 to initiate at least one action (308) based on the determination that the nighttime ventricular filling pressure measurements were larger than the daytime measurements (306).

For example, processor 106 may cause communications module 122 to alert patient 10 or a clinician of the abnormal ventricular filling pressure pattern or transfer data showing the pattern of ventricular filling pressures to the clinician or other qualified person for evaluation. In some examples processor 106 may initiate a treatment or alter a dosage in a course of treatment performed by AMD 104. After initiating at least one action based on the determination, pressure monitor 100 and sensor 102 may continue to measure a physiological parameter indicative of ventricular filling pressure, enabling pressure monitor 100 to monitor the effects of the adjustment of therapy, detecting amelioration or worsening of the physiological factors contributing to HF in patient 10. If physiological factors improve, pressure monitor 100 may gradually revert treatment, such as that dispensed by AMD 104, to prior levels. If physiological factors worsen, pressure monitor 100 may further increase treatment levels, alter the form or timing of the treatment, or issue new alerts to patient 10 or a clinician indicating the situation.

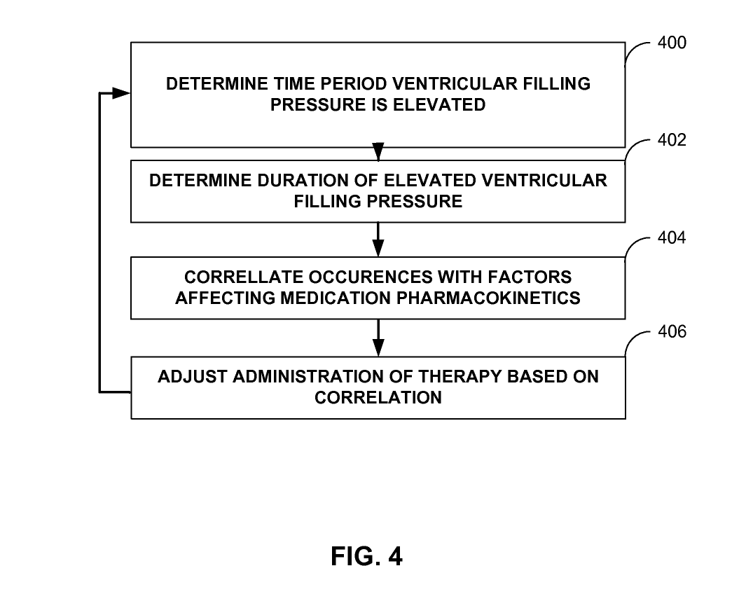

FIG. 4 is a flow diagram illustrating an example method of monitoring a physiological parameter of a patient and controlling treatment of HF. The method may include determining a time period when the ventricular filling pressure is elevated (400), determining the duration of the elevated ventricular filling pressure (402), correlating the occurrences of elevated ventricular filling pressure with factors affecting medication pharmacokinetics (404), and adjusting the administration of therapy based on the correlation (406).

Pressure monitor 100 may determine, through activity sensor 118 or clock 120, a time period when the night time ventricular filling pressure is elevated (400). As described with respect to FIG. 3, pressure monitor 100 may communicate with sensor 102, monitoring one or more physiological parameters representative of the ventricular filling pressure. Pressure monitor 100 may determine the time at which the elevated nighttime ventricular filling pressure occurred using clock 120. Further, pressure monitor 100 may determine the duration of the elevated night time filling pressure (402) using clock 120.

Pressure monitor 100 may correlate the time and duration of the elevated night time ventricular filling pressures to the timing of factors affecting the pharmacokinetics of a medication. Activities or conditions of the patient that may affect the pharmacokinetics of a medication may be sensed via one or more of pressure monitor 100 or sensor 102, or determined based on user input, e.g., in the context of a medical diary (404). The user input may be received by a programmer for monitor 100 or another computing device in communication with monitor 100. Activity level, food intake, posture, medication or therapy type, and similar factors may affect the ability of a medication or other therapy to address the factors that may lead to patient 10 experiencing HF symptoms. Many of these events take place at regular points in the daily life of a patient. For example, a patient with a job tends to structure meals, physical activity, and sleep periods around the work schedule. Because these activities are regular, detection of factors that may indicate a worsening of symptoms, such as elevated night time ventricular filling pressure, occurring at a regular time and duration may be linked with a regularly occurring patient lifestyle activity that may affect the pharmokinetics of a particular medication.

For example, a prescribed medication may be sensitive to food ingestion and elevated night time ventricular filling pressures may detected early in the evening closely following the time appropriate for the consumption of a meal. In some examples, pressure monitor 100 may receive information regarding the time of consumption of the meal from an external device. Pressure monitor 100 may link the time the medication is administered with the elevated night time ventricular filling pressure and compensate, by, for example, administering the medication earlier to avoid the meal.

Pressure monitor 100 may adjust the administration of the therapy (406) to compensate for the pharmacokinetic factors correlated with the elevated night time ventricular filling pressures detected by sensor 102 and pressure monitor 100. In the previous example, delivering blood pressure medication immediately after the patient consumed food and prior to sleep may have allowed temporary elevation of the nighttime ventricular filling pressure of the patient. Adjusting the timing of the delivery of medication, such as before the meal rather than after, may normalize the circadian variation of the ventricular filling pressure. In some examples, pressure monitor 100 may communicate instructions to AMD 104, causing AMD 104 to adjust the treatment of patient 10, for example increasing or decreasing the amount of medication dispensed, or duration of therapy, or when the medication or therapy should be administered, allowing the treatment to have greater effect by compensating for the medication pharmacokinetics and the lifestyle of patient 10.

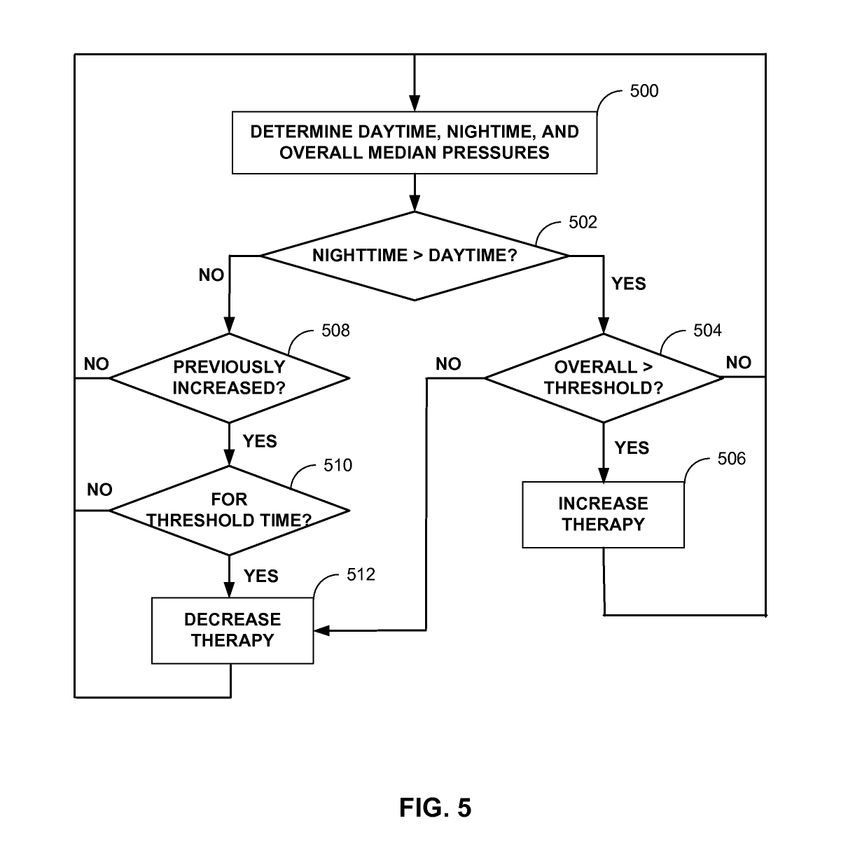

FIG. 5 is a flow diagram illustrating an example method of controlling treatment for HF based on comparing daytime and nighttime measured values of a physiological parameter. The example method may be implemented by a system, such as system 2A or 2B (FIGS. 1A and 1B).

The example method includes determining representative daytime, nighttime, and overall values of a physiological parameter indicative of ventricular filling pressure (500). The representative daytime and nighttime values may be individual values, or mean or median values, and the overall value may be a mean or median of daytime and nighttime values. The method further includes comparing the nighttime values to the daytime values (502).

If the nighttime values are greater, the method further includes determining whether the overall median value is greater than a threshold (504), e.g., indicating that overall pressure is high enough to warrant treatment to lower ventricular filling pressure. If the overall value representative of ventricular filling pressure is greater than the threshold, then therapy may be increased (506). In some examples, processor 106 may instruct AMD 104 to increase the dosage of medication intended to treat the physiological factors of HF, or cause AMD 104 to administer a therapy intended to treat HF. Processor 106 may also cause a notification to be sent to patient 10 or a clinician indicating the abnormal ventricular filling pressure pattern. Processor 106 may record the abnormal ventricular filling pressures for later diagnosis by the clinician.

The treatment may be a nighttime therapy to lower nighttime ventricular pressure, which may not be appropriate of overall (day and night) pressure is already below the threshold despite the nighttime pressure being greater than the daytime pressure. Common treatments for HF include medications to reduce blood pressure. If the ventricular filling pressures are already low, introducing or increasing the dosage of medications intended to reduce blood pressure may be undesired, even if the night time ventricular filling pressure is larger at night than during the day. If the overall value representative of ventricular filling pressure is not greater than the threshold, therapy may be maintained at a current level, or decreased (512), e.g., to allow the ventricular filling pressures to increase to safer levels. Furthermore, if the nighttime ventricular filling pressures are the same as, or lower than the representative daytime ventricular filling pressure, the system may check to see if therapy has been previously increased (508). If so, then the system may check to see if the current level of therapy has been maintained for a threshold time (510). If the time threshold has been exceeded, the system may decrease or maintain the therapy (512).

If ventricular filling pressures are greater during the day then at night, processor 106 of pressure monitor 100 may determine whether the therapy was previously increased (508). If the therapy has not been increased, system 2 may continue to monitor one or more physiological parameters representative of the ventricular filling pressure of patient 10. If the therapy was previously increased or adjusted, processor 106 may verify that the increased or adjusted therapy/dosage has been maintained for threshold time, allowing the medication or therapy to reach full effect and for temporary conditions to pass. After the therapy has been maintained for the threshold time, processor 106 may instruct AMD 104 or other device to reduce therapy (512). In some examples this reduction may be incremental, e.g., by a fixed amount or to a previous level of therapy. Alternatively, therapy may be reduced to the original amount or ceased completely. Pressure monitor 100 and sensor 102 continue to monitor the ventricular filling pressure of patient 10 to verify that the reduced treatment levels are effective at preventing abnormal nighttime ventricular filling pressures in patient 10. Reductions in therapy may take place periodically to ensure that the physiological factors contributing to HF in patient 10 have not changed over the course of the therapy. In general, if therapy is neither increased (506) or decreased (512), e.g., due to the overall pressure value being less than the threshold (“NO” of 504) when the nighttime pressure is greater than the daytime pressure, the therapy not having been previously increased when the daytime pressure is greater than the nighttime pressure (“NO” of 508), or because a threshold time period from a previous therapy increase has not been met (“NO” of 510), then the therapy is maintained at its current level, which may be a baseline or user-prescribed level.

Pressure monitor 100 may determine the timing or magnitude of the modification to the therapy, such as increasing therapy (506), based on the timing or duration of the nighttime filling pressure exceeding the daytime filling pressure, as well as the magnitude of the difference between the nighttime and daytime values. In some examples, pressure monitor 100 may determine whether modification of daytime therapy, nighttime therapy, or both is warranted based on these factor regarding the relationship of the nighttime and daytime filling pressures. The magnitude of the overall filling pressure, e.g., daily mean value of the physiological parameter indication of ventricular filling pressure, may also be used to determine the magnitude of the change to the therapy.

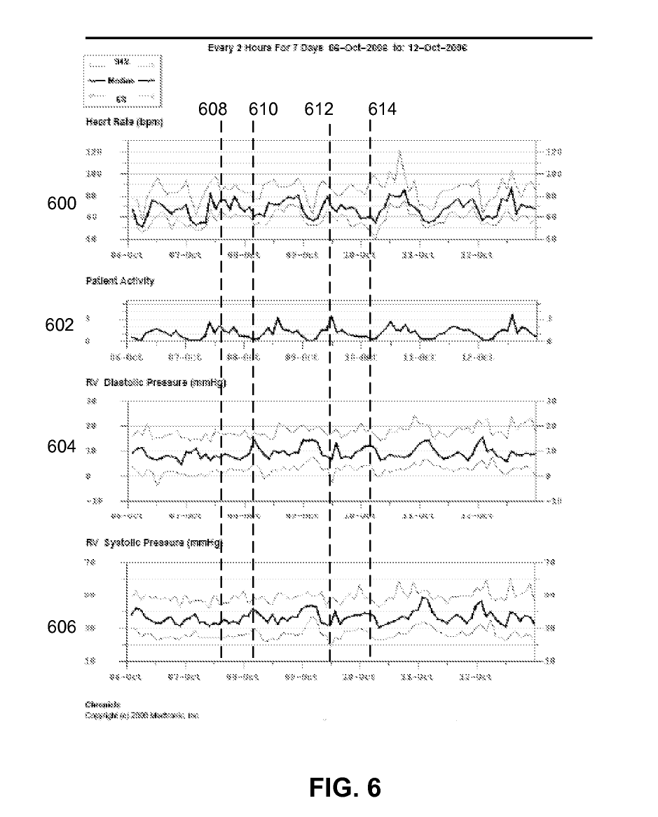

FIG. 6 is a chart illustrating an example patient exhibiting an atypical pattern of cardiac pressures. Chart 600 graphs the heart rate of a patient. Chart 602 depicts patient activity levels. Chart 604 displays right ventricle diastolic pressure. Chart 606 displays right ventricle systolic pressure. The data points comprising each chart were taken every two hours over a seven day stretch. Lines 608, 610, 612, and 614 mark points in two day-night cycles as indicated by the activity levels of the patient.

Typically, patients have increases in filling pressure during the active daytime hours, due to mechanisms acting to adjust filling pressures to accommodate the cardiovascular stresses encountered with normal activities of daily life. These increases in daytime filling pressures are typically seen, even though gravitational forces associated with upright body position, taken alone, will be acting to decrease filling pressures as fluid shifts away from the thoracic vasculature to the gravity dependent body areas like the gut and lower extremities. FIG. 6 depicts atypical patterns of circadian filling pressures in a patient; the nighttime filling pressures are higher than their active daytime filling pressures. Potentially, the elevated nighttime filling pressure produce a variety of undesired patho-physiological responses such as increased load on the heart (left and right ventricles), increased filtration of fluid to extravascular compartments (pulmonary congestion), and chronic changes in pulmonary vascular reactivity. Additionally, since the patient is recumbent and inactive at this time, the opportunity to delivery additional vasodilator therapy may be considered since the risk of symptomatic systemic hypotension is lower at this time.

Comparing the heart rate of chart 600 to the activity level of chart 602 demonstrates the close relationship between heart rate and activity level. Elevated heart rates match elevated levels of patient activity. For example, the three peaks of activity level centered on line 608 are matched by three peaks of heart rate that occur at the same time. The heart rate slows around line 610 and is matched by a corresponding reduction the patient activity level. The heart rate, activity level, and time that the measurements about line 610 were made indicate that the patient was at rest, likely asleep and in a supine or prone position. Similarly the activity levels at lines 612 and 614 are matched by corresponding patterns in the heart rate. This demonstrates that activity level and heart rate may be a predictor of patient posture. In this patient, the lowest ebbs in heart rate and activity level occur regularly at times just past midnight. Given the reduced activity level and heart rate and the time at which these reductions occur, it is very likely that these reductions are caused by the patient sleeping, and therefore the patient being in a supine or prone position. The comparison also demonstrates the effectiveness of heart rate as indicator of patient activity level. Being able to use the heart rate as a measure of activity level may allow pressure monitor 100 or other device that measures activity level to be simplified, in that instead of a motion sensor an electrode which detects the electrical impulses of the heart may be used.

Comparing charts 604 and 606 indicate a close agreement between the patterns of diastolic and systolic pressure readings in the right ventricle of the heart of the patient. While the magnitude of the systolic and diastolic pressures are different, the patterns followed by the median of the two pressures are very similar. For example, at line 608, both the systolic and diastolic pressure exhibit three small peaks preceded by a larger double peak. At line 610, both pressure curves exhibit a large increase followed by two secondary peaks.

During the daylight hours, the systolic and diastolic pressures of the right ventricle follow patterns similar to the heart and activity level of the patient. At line 608, the three smaller peaks in the systolic and diastolic pressure are evident in both the activity level and the heart rate. The large peak preceding the three smaller peaks in the systolic and diastolic pressure charts 604 and 606 occurs in a night time period when the posture of the patient exhibits an increased effect. Further, the sharp single peak in the systolic and diastolic pressure curves at line 612 is reflected in activity levels and heart rate of the patient.

Comparing the day and night systolic and diastolic pressures demonstrate the abnormalities of the cardiac cycle of the patient. Comparing the right ventricular pressures between lines 608 and 610 demonstrate a marked increase in pressure despite the reduction in the heart rate and activity level of patient indicating that the patient is asleep and therefore supine or prone. A similar increase in cardiac pressure occurs between the day time period at line 612 and the night time period at line 614. Given that ventricular filling pressure should decrease when the patient is at rest, the abnormal patterns in the systolic and diastolic pressure of the right ventricle may indicate a worsening of the HF of the patient and may require additional treatment, such as an increase in dosage of vasodilators or diuretics. An evaluation of the patient by a clinician may also allow the treatment of the patient to be adjusted to better alleviate the physiological factors contributing to HF. The record of these abnormal cardiac pressure cycles may allow the clinician to make a more informed diagnosis.

The techniques described in this disclosure may be implemented, at least in part, in hardware, software, firmware, or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various techniques described in this disclosure. In addition, any of the described units, modules or components may be implemented together or separately as discrete but interoperable logic devices. Depiction of different features as modules or units is intended to highlight different functional aspects and does not necessarily imply that such modules or units may be realized by separate hardware, firmware, or software components. Rather, functionality associated with one or more modules or units may be performed by separate hardware, firmware, or software components, or integrated within common or separate hardware, firmware, or software components.

The techniques described in this disclosure may also be embodied or encoded in a computer-readable medium, such as a computer-readable storage medium, containing instructions. Instructions embedded or encoded in a computer-readable medium, including a computer-readable storage medium, may cause one or more programmable processors, or other processors, to implement one or more of the techniques described herein, such as when instructions included or encoded in the computer-readable medium are executed by the one or more processors. Computer readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or other computer readable media. In some examples, an article of manufacture may comprise one or more computer-readable storage media.

Various examples have been described. However, one of ordinary skill in the art will appreciate that various modifications may be made to the described examples without departing from the scope of the claims. For example, although described primarily with respect to examples in which a pressure monitor 100 controls a modification to therapy delivery by an AMD 104 in response to a comparison of nighttime and daytime values of physiological parameters indicative of ventricular filling pressure, in other examples pressure monitor may control a different implanted or external therapy device, such as ultrafiltration device, or a pacemaker.

With respect to the control of ultrafiltration therapy, pressure monitor 100 may modify various parameters of ultrafiltration based on the timing and/or duration of nighttime filling pressures exceeding daytime filling pressures, e.g., how long, in days, the nighttime pressure has been greater than the daytime pressure, as well as the magnitude of the difference(s) between the nighttime and daytime values indicative of filling pressure. The timing, duration or relative magnitude of the nighttime pressure exceeding the daytime pressure may be used to determine if the ultrafiltration therapy is more efficacious when applied during the night, when filling pressures are most elevated, rather than during the day. Pressure monitor 100 may control the schedule of ultrafiltration based on such factors. For example, pressure monitor 100 may control the number of ultrafiltration sessions per period, e.g., per night, as well as the number of periods, e.g., nights, for which ultrafiltration should be delivered. Serial ultrafiltration periods or sessions may continue at relatively low rates for several days or nights until the day-to-night pressure difference is reduced. In some examples, pressure monitor 100 may determine an amount of fluid to remove from the body of a patient based on both the magnitude of difference in the filling pressure from nighttime to daytime, as well as an overall filling pressure value, e.g., a daily mean of values indicative of filling pressure.

Modification of a therapy delivered by a pacemaker may include modification of one or more parameters of pacing, such as rate, rate response, mode, or vector, or to provide different values for parameters during nighttime then daytime. Modification of CRT may include one or more of modification of A-V or V-V intervals, or the selection of different electrodes for delivery of RV or LV pacing, or the selection of different values for such parameters during nighttime then daytime. In some examples, pressure monitor 100 may be embodied in an implantable pacemaker, which may include cardioversion and defibrillation capabilities, and which may provide CRT to patient 10.

Although described herein primarily in the context of modification of treatment factors that may lead to worsening heart failure in response to detecting that nighttime ventricular filling pressures are greater than daytime ventricular filling pressures, other examples may include modification of treatment of other maladies based on a comparison of daytime and nighttime ventricular filling pressures. In some other examples, nighttime filling pressures may be lower—unusually lower—than either the normal nighttime pressures or daytime pressures, and techniques according to the invention may include initiating or modifying a therapy in response to such a condition. These and other examples are within the scope of the following claims.

1. A system comprising:
a sensor configured to measure a plurality of values of a physiological parameter indicative of ventricular filling pressure of a patient; and
a processor configured to:
for each of the measured values of the physiological parameter, categorize the value as one of a daytime value or a nighttime value,
compare one or more of the daytime values to one or more of the nighttime values, and
if the nighttime values are larger than the daytime values, direct a modification of delivery of a therapy for treatment of heart failure.

2. The system of claim 1, further comprising:
a communications module,
wherein the processor activates the communications module to provide notification to the patient or a designated clinical provider when the nighttime values are larger than the daytime values.

3. The system of claim 1, further comprising:
an automated medication dispenser,
wherein the processor provides input to the automated medication dispenser to adjust a dosage of a medication dispensed by the automated medication dispenser to the patient when the nighttime values are larger than the daytime values.

4. The system of claim 3, wherein the processor is configured to intermittently withhold therapy and verify that the nighttime values are larger than the daytime values without the medication dispensed by the automated mediation dispenser.

5. The system of claim 3, wherein the medication comprises a vasodilator.

6. The system of claim 1, further comprising:
an ultrafiltration device,
wherein the processor provides input to the ultrafiltration device to adjust a dosage of ultrafiltration provided to the patient by the ultrafiltration device when the nighttime values are larger than the daytime values.

7. The system of claim 6, wherein the dosage of ultrafiltration comprises at least one of the time of day to institute ultrafiltration, the amount of a fluid to remove, the rate of removal of the fluid, the duration of the ultrafiltration, and the number of times ultrafiltration is repeated.

8. The system of claim 1, further comprising:
a cardiac electrical stimulus device,
wherein the processor provides input to the cardiac electrical stimulus device to adjust therapy provided to the patient by the cardiac electrical stimulus device when the nighttime values are larger than the daytime values.

9. The system of claim 8, wherein the processor controls the cardiac electrical stimulus device is configured to provide therapy with different parameters at night than during the day when the nighttime values are larger than the daytime values.

10. The system of claim 1, wherein the sensor comprises a pressure sensor.

11. The system of claim 1, further comprising:
a position sensor,
wherein the processor categorizes the values as one of a daytime value or a nighttime value based on the position of the body of the patient detected via the position sensor.

12. The system of claim 1, further comprising:
an activity monitor,
wherein the processor categorizes the values as one of a daytime value or a nighttime value based on an activity level of the patient detected via the activity sensor.

13. The system of claim 1, wherein the processor is further configured to:
determine a representative overall value of the physiological parameter based on the measured values of the physiological parameter,
compare the overall value of the physiological parameter to a threshold, and
if the nighttime values are larger than the daytime values and the overall value is greater than the threshold, direct the modification of delivery of a therapy for treatment of heart failure.

14. A method comprising:
measuring a plurality of values of a physiological parameter indicative of ventricular filling pressure of a patient by a sensor;
with a processor, for each of the measured values of the physiological parameter, categorizing the value as one of a daytime value or a nighttime value;
with the processor, comparing one or more of the daytime values to one or more of the nighttime values; and
with the processor, if the nighttime values are larger than the daytime values, directing a modification of delivery of a therapy for treatment of heart failure.

15. The method of claim 14, further comprising:
providing notification to the patient or a clinician when the nighttime values are larger than the daytime values.

16. The method of claim 14, further comprising:
adjusting a dosage of a medication dispensed to the patient by an automated medication dispenser when the nighttime values are larger than the daytime values.

17. The method of claim 16, further comprising:
intermittently withholding therapy and verifying that the nighttime values are larger than the daytime values without the medication dispensed by the automated mediation dispenser.

18. The method of claim 16, wherein the medication comprises a vasodilator.

19. The method of claim 14,
wherein the sensor that measures a plurality of values of a physiological parameter indicative of ventricular filling pressure of a patient comprises a pressure sensor.

20. The method of claim 19, wherein the pressure sensor is disposed in a right ventricle.

21. The method of claim 19, wherein the pressure sensor is disposed in a pulmonary artery.

22. The method of claim 14, further comprising:
determining a representative overall value of the physiological parameter based on the measured values of the physiological parameter; and
comparing the overall value of the physiological parameter to a threshold,
wherein directing the modification of delivery of a therapy for treatment of heart failure comprises directing the modification if the nighttime values are larger than the daytime values and the overall value is greater than the threshold.

23. The method of claim 14, further comprising
correlating the measured values of the physiological parameter with one or more factors affecting the pharmacokinetics of the patient; and
adjusting the at least one action based on correlation with the one or more factors affecting the pharmacokinetics of the patient.

24. A system comprising:
means for measuring a plurality of values of a physiological parameter indicative of ventricular filling pressure of a patient;
means for, for each of the measured values of the physiological parameter, categorizing the value as one of a daytime value or a nighttime value;
means for comparing one or more of the daytime values to one or more of the nighttime values; and
means for, if the nighttime values are larger than the daytime values, directing a modification of delivery of a therapy for treatment of heart failure.

25. A system comprising:
a sensor configured to measure a plurality of values of a physiological parameter indicative of ventricular filling pressure of a patient; and
a processor configured to:
for each of the measured values of the physiological parameter, categorize the value as one of a daytime value or a nighttime value,
compare one or more of the daytime values to one or more of the nighttime values, and
direct a modification of delivery of a therapy to the patient based on the comparison.
